(19) United States
(12) Patent Application Publication
(10) Pub. No.: US 2025/0388494 A1
(43) Pub. Date: Dec. 25, 2025

(54) DEVICE AND METHOD FOR THE PRODUCTION OF PERMEATE, IN PARTICULAR FOR DIALYSIS THERAPY

(71) Applicant: B. Braun Avitum AG, Melsungen (DE)

(72) Inventors: Nino Lieb, Kassel (DE); Philipp Odernheimer, Melsungen (DE)

(21) Appl. No.: 19/245,371

(22) Filed: Jun. 22, 2025

(30) Foreign Application Priority Data
Jun. 25, 2024 (DE) 10 2024 117 910.4

(51) Int. Cl. C02F 1/44; A61M 1/16; B01D 61/02; B01D 61/12; B01D 61/14; B01D 61/22; B01D 61/58; C02F 103/02

(52) U.S. Cl. CPC C02F 1/441; A61M 1/1601; A61M 1/1696; B01D 61/12; B01D 61/22; B01D 61/58; C02F 1/444; A61M 2205/3337; A61M 2205/36; B01D 61/025; B01D 61/14; B01D 2313/903; C02F 2103/026; C02F 2209/03; C02F 2209/40; C02F 2209/42; C02F 2301/046

(57) ABSTRACT

A water treatment plant or system for the production of permeate includes a reverse osmosis stage and a permeate stage. The reverse osmosis stage can include a piping system having a supply line for process input water, a reverse osmosis pump, and a reverse osmosis tank. The reverse osmosis tank has an inlet for process input water coming from the reverse osmosis pump, a membrane, and an outlet for permeate. The permeate stage, which can have a line system forming a circuit, can include a permeate tank, a permeate pump, and a tapping point for connecting at least one consumer or user of permeate. The reverse osmosis stage and the permeate stage can be connected to one another via a connecting line in such a way that the reverse osmosis stage feeds the permeate stage with permeate. The circuit of the permeate stage can include a sterile filter.

### CROSS REFERENCE TO RELATED APPLICATION

This application claims priority under 35 U.S.C. § 119 to German Application No. 10 2024 117 910.4, filed on Jun. 25, 2024, the content of which is incorporated by reference herein in its entirety.

### FIELD

The present disclosure relates to a water treatment system for the production of permeate, with a focus on use in dialysis therapy. It also relates to an associated method.

### BACKGROUND

The state of the art includes various approaches to water treatment, in particular those based on the principle of reverse osmosis. These processes are widely used, particularly in dialysis applications, to produce high purity water and therefore high quality permeate. However, existing systems can present challenges in terms of efficiency, control and quality optimization.

In particular, so-called stand-alone systems, which are often set up directly at the treatment site for dialysis, have a comparatively high energy requirement due to the constant operation of the pump(s) required for reverse osmosis. Noise pollution for the patient can also be significant.

### SUMMARY

The object of the present disclosure is to provide a water treatment plant of the type mentioned, which produces permeate of high purity in an energy-efficient and low-noise manner and at the same time as reliably as possible. In addition, an associated process for the production of permeate is to be disclosed.

With regard to the device, the said task is solved according to the present disclosure by a water treatment plant for the production of permeate, in particular for dialysis therapy, comprising:
a reverse osmosis stage with a piping system comprising a supply line for process input water, a reverse osmosis pump and a reverse osmosis tank (also called pressure pipe), the reverse osmosis tank having an inlet for the process input water coming from the reverse osmosis pump, a membrane and an outlet for permeate,
a permeate stage with a pipe system forming a circuit, which comprises a permeate tank, a permeate pump and a tapping point for connecting at least one consumer or user of permeate,
wherein the reverse osmosis stage and the permeate stage are connected to one another via a connecting line in such a way that the reverse osmosis stage feeds the permeate stage with permeate, and wherein the circuit of the permeate stage comprises a sterile filter.

The term “stage” is preferably understood here generally in the sense of “unit” or “device” and does not exclude the possibility that the reverse osmosis stage itself may also have a multi-stage structure.

The present disclosure thus aims to feed the permeate produced in the reverse osmosis stage to a permeate tank for intermediate storage, from where it can be kept in circulation by means of a separate permeate pump with comparatively low energy consumption. The reverse osmosis pump in the reverse osmosis stage, which requires more energy than the permeate pump, can then be switched off for longer periods of time until a new batch of permeate has to be produced after it has been taken by a consumer.

In other words, the water treatment system automatically adapts to demand by controlling (switching on and off) the reverse osmosis pump depending on the fill level in the permeate tank. This helps to use resources efficiently and adapt operation to actual requirements. This also ensures a predominantly low-noise performance of the system, which improves the comfort and well-being of the patient.

According to the present disclosure, the circuit of the permeate stage further comprises a sterile filter, which is preferably arranged downstream of the permeate pump and upstream of the permeate tank. The integration of a sterile filter in the circuit of the permeate stage ensures additional safety and purity of the permeate produced, in that the sterile filter separates or retains microorganisms which could potentially be in the permeate circuit.

Advantageous embodiments are the subject of the following detailed description.

Furthermore, it is preferable if the tapping point for the consumers that can be connected, for example by means of hose couplings, is arranged downstream of the sterile filter and upstream of the permeate tank, whereby an overflow valve or a flow limiter is advantageously arranged between the tapping point and the permeate tank in order to ensure the desired or required pressure at the tapping point.

Preferably, there is a system control that switches the reverse osmosis pump on and off depending on the fill level in the permeate tank. As already mentioned, the reverse osmosis pump only runs when there is a real need for it.

To implement this type of demand-based control, a pressure-based fill level detection system can be provided for the permeate tank, in which, for example, the hydrostatic pressure of the permeate column in the permeate tank is measured and the fill level or fill volume is determined on this basis.

In one possible variant, the permeate pump is pressure-controlled depending on the pressure in the circuit of the permeate stage. This means that the pump output or speed of the permeate pump is automatically adapted to the prevailing pressure in the circuit or is regulated so that a predetermined target pressure is reached and maintained. This pressure-regulated function enables precise control of the permeate throughput by modulating the permeate pump according to the pressure conditions in the system. This not only helps to improve process stability, but also to avoid undesirable pressure fluctuations. However, the main aim is to save electrical energy and reduce noise emissions, as the speed of the permeate pump can be reduced when no permeate is being drawn off.

In this variant, it is advantageous if a pressure sensor used for regulation is arranged in the circuit between the tapping point and a flow limiter arranged upstream of the permeate tank.

In an alternative variant, the permeate pump is volume flow-controlled depending on the flow rate through the circuit of the permeate stage. The volume flow-controlled permeate pump enables precise adjustment of the pump speed with the aim of stabilizing the throughput in the circuit. This is particularly advantageous for adapting the permeate flow to the requirements of consumers or customers and ensuring optimum utilization of the permeate produced. Here too, however, the focus is on saving electrical energy.

In this variant, it is advantageous if a volumetric flow sensor used for control is arranged in the circuit between the tapping point and an overflow valve arranged upstream of the permeate tank.

In one possible version, the permeate tank is also designed as a pressure expansion vessel. This ensures precise pressure control in the system. The pressure expansion tank primarily serves as a buffer for permeate so that the connected dialysis machine does not run dry. It also helps to minimize unwanted pressure fluctuations and increase the service life of the components.

In a preferred embodiment, the connecting line between the reverse osmosis stage and the permeate stage is designed in such a way that the entire permeate production of the reverse osmosis stage is fed into the circuit of the permeate stage. This means that the reverse osmosis stage preferably does not have its own extraction points, but the permeate is extracted exclusively via the circuit of the permeate stage.

In a possible further development, the reverse osmosis stage has a heater for the process inlet water that works on the principle of a continuous-flow heater, which is preferably arranged between the reverse osmosis pump and the reverse osmosis tank. This allows the water to be heated for the purpose of hot cleaning or hot disinfection of all downstream line sections of the reverse osmosis stage and the permeate stage.

If the reverse osmosis stage has a recirculation line for concentrate retained by the membrane, this mainly serves to increase the efficiency in terms of water consumption, as the water can enter the filtration process again. Furthermore, premature blocking of the membrane is avoided. The re-injection of concentrate from the recirculation line into the main line from the reverse osmosis pump to the reverse osmosis tank can be carried out using a Venturi nozzle, for example.

In a further advantageous embodiment, the reverse osmosis stage has a storage tank for the intermediate storage of process input water. The integration of a storage tank in the reverse osmosis stage enables the intermediate storage of process input water. This is particularly advantageous in order to compensate for fluctuations in water demand or in the water supply and to ensure continuous permeate production, at least temporarily.

The reverse osmosis stage and the permeate stage can be structurally separate and installed at different locations (e.g. with a correspondingly long connecting line), but they can also be structurally integrated in a compact device, for example in a single-station reverse osmosis system that is installed directly next to a dialysis station. For certain applications, it may also be advantageous to separate the two units in the housing in order to reduce noise and/or to provide a modular solution that can be stowed away more flexibly individually than the entire apparatus in one housing. In general, it is advantageous to keep the line between the reverse osmosis system and the dialysis machine short in order to minimize the standing water volume.

The present disclosure also provides a method for producing permeate, in particular for dialysis therapy. Preferably, a water treatment plant of the type described above is used for this purpose. The method is characterized in that permeate is produced according to the principle of reverse osmosis, in that process input water is pressed through a membrane by means of a reverse osmosis pump, the permeate produced is then fed, preferably completely, into a permeate tank and is circulated by means of a permeate pump in a circuit connected to the permeate tank, wherein possible consumers or users of permeate can be connected to the circuit, and wherein the permeate in the circuit is passed through a sterile filter.

Advantageously, the reverse osmosis pump is switched off when the level of permeate in the permeate tank exceeds a defined value and is only switched on again when the level of permeate in the permeate tank falls below a defined value.

The tasks, features, variants and advantages mentioned for the device apply mutatis mutandis to the method and vice versa.

### BRIEF DESCRIPTION OF THE DRAWINGS

Various embodiments of the present disclosure are explained in more detail below with reference to the accompanying drawings.

FIG. 1 shows a simplified hydraulic circuit diagram of a reverse osmosis system for the production of permeate for hemodialysis therapy according to the state of the art (Prior Art);

FIG. 2 shows a water treatment plant according to the present disclosure for the production of permeate for hemodialysis therapy, wherein the plant is preferably designed as a single-station plant, and wherein the plant is equipped with a reverse osmosis stage and a permeate stage with a separate permeate tank;

FIG. 3 shows a water treatment plant of the type shown in FIG. 2 with a volume-flow-controlled permeate pump;

FIG. 4 shows a water treatment plant of the type shown in FIG. 2 with pressure-controlled permeate pump;

FIG. 5 shows a water treatment plant of the type shown in FIG. 2 with an additional storage tank in the reverse osmosis stage;

FIG. 6 shows a water treatment plant of the type shown in FIG. 2 with additional hot cleaning option;

FIG. 7 shows an extension of the water treatment plant based on FIG. 6 with recirculation option for permeate;

FIG. 8 shows a water treatment plant of the type shown in FIG. 2 with a pressure expansion vessel in the permeate stage; and

FIG. 9 shows a water treatment plant of the type shown in FIG. 2 with a venturi nozzle for concentrate recirculation in the reverse osmosis stage.

Elements that are identical or have the same effect are marked with the same reference symbols in all figures.

### DETAILED DESCRIPTION

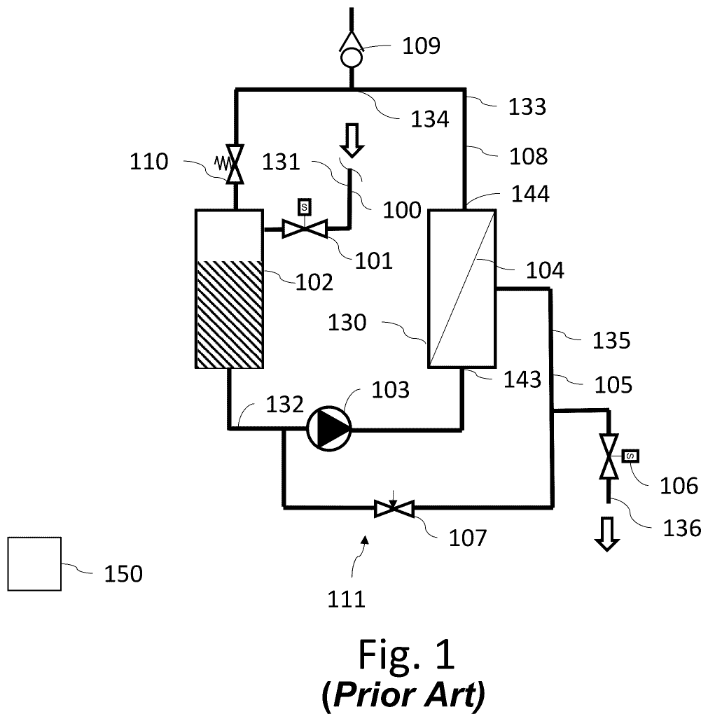

FIG. 1 provides an overview of a reverse osmosis system 111 for the production of permeate for hemodialysis therapy according to the state of the art in the form of a hydraulic block diagram.

Essential components of the reverse osmosis system 111 are a storage tank 102 for process input water 100 and a reverse osmosis tank 130 with a semi-permeable membrane 104 (reverse osmosis membrane), which are integrated into a hydraulic circuit via a pipe system. A supply line 131 for process input water 100 (also known as soft water) is connected to the feed tank 102. The supply of process input water 100 to the supply tank 102 can be controlled via a solenoid valve 101 or the like connected to the supply line 131. The feed tank 102 and the reverse osmosis tank 130 are connected to each other via a connecting line 132 in such a way that, during operation, process input water 100 is conveyed from the feed tank 102 into the reverse osmosis tank 130 by means of a reverse osmosis pump 103 connected into the connecting line 132, where it is pressed through the membrane 104. Accordingly, the water enters the reverse osmosis tank 130 through the inlet 143, which is also referred to in technical circles as a “pressure pipe” or “membrane pressure vessel”, then passes through the membrane 104 and exits the reverse osmosis tank 130 through the outlet 144. In this process, according to the principle of reverse osmosis, impurities contained in the process input water 100, primarily in the form of ions, are retained on the concentrate side (dirty side) of the reverse osmosis vessel 130, i.e. upstream of the membrane 104. In concentrated form, the impurities are also referred to as concentrate 105.

Purified water or permeate 108 emerging from the membrane 104 on the permeate side (clean side) is fed through a ring line 133 to a number of consumers or recipients, each of which can be connected to the ring line 133 at a tapping point 134 via a coupling 109. Permeate 108 that is not consumed or removed is circulated back into the feed tank 102 via the ring line 133. An overflow valve 110 connected downstream of the coupling 109 in the ring line 133 opens when a set holding pressure is reached or exceeded, thereby ensuring a minimum pressure at the tapping point 134.

In a preferred case, the reverse osmosis vessel 130 may be a pressure tube into which the membrane 104 is inserted. Such a pressure pipe preferably has openings for inserting and exchanging the membrane modules, which distinguishes it from simple pipes. The pressure pipes are also often larger (in diameter) than the normal line cross-sections due to the membrane geometry.

Furthermore, at least partial recirculation of concentrate 105 can be provided on the concentrate side of the circuit. For this purpose, a (concentrate) recirculation line 135 is connected to the concentrate side of the reverse osmosis tank 130, which opens into the connecting line 132 at the other end upstream of the reverse osmosis pump 103. A needle valve 107 or the like (flow limiter) connected into the recirculation line 135, preferably with adjustable flow rate, limits the recirculation and prevents a fluidic short circuit. The recirculation prevents or at least delays the concentrate 105 from sticking to the membrane 104. Primarily, however, the clogging of membranes is prevented more by the fact that water is led past the membrane at all. It does not matter whether the water is fed into the connecting line or into the drain 136.

Furthermore, a drain line, or drain 136 for short (or “discard”) branches off from the recirculation line 135, wherein the drain 136 is closed by a controllable solenoid valve 106 during normal operation of the reverse osmosis system 111. By opening the solenoid valve 106, concentrate 105 can be discarded from the reverse osmosis tank 130 or the reverse osmosis circuit as required. When discarding concentrate (which has many ions compared to the process input water), the missing volume is replaced by process input water from the receiver tank 102 (with fewer ions). This reduces the total number of ions in the process water in the circuit.

A disadvantage of such systems is the high energy consumption due to the continuous reverse osmosis. In addition, noise pollution for the user due to the constantly running reverse osmosis pump 103 must also be mentioned. This is particularly relevant for stand-alone reverse osmosis systems, as these devices are usually positioned close to the patient.

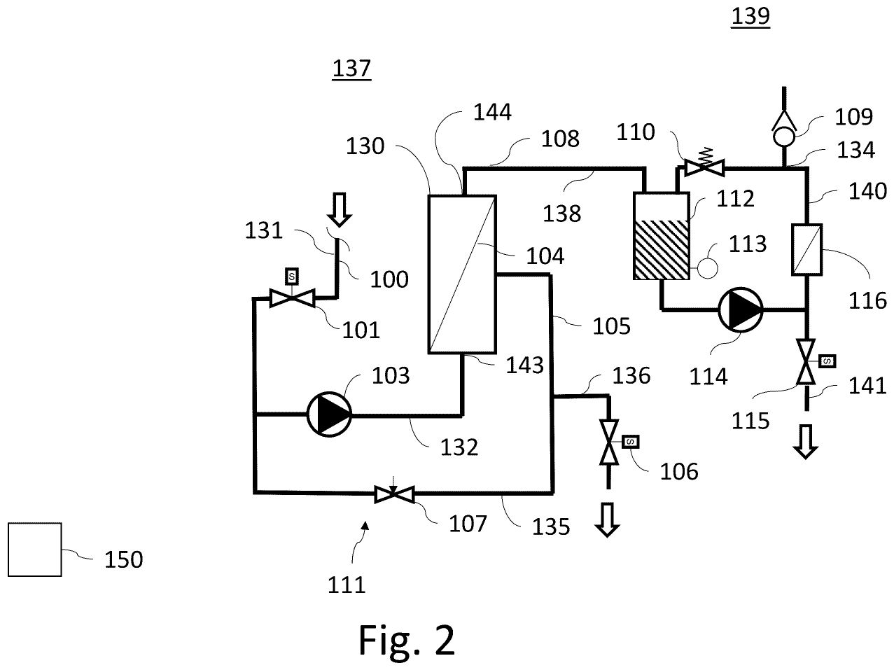

To avoid such problems, the water treatment system shown in a schematic overview in FIG. 2, further referred to collectively as reverse osmosis system 111, comprises two subunits. The first sub-unit, which may also be referred to as the reverse osmosis stage 137 or reverse osmosis unit or RO stage (RO=reverse osmosis), draws process input water 100 via a supply line 131 into which a solenoid valve 101 is connected. Downstream of the solenoid valve 101, the supply line 131 merges into the connecting line 132 known from FIG. 1, which is connected at the other end to the reverse osmosis tank 130. In contrast to the system according to FIG. 1, a storage tank 102 for intermediate storage of the process input water 100 is not necessarily provided here, but may be present in a modified variant (see below). The reverse osmosis pump 103 is inserted into the connecting line 132. As in the prior art, the process input water 100 is thus pumped by the reverse osmosis pump 103 into the reverse osmosis tank 130 with the membrane 104 and pressed through it. In accordance with the principle of reverse osmosis, impurities contained in the process input water 100, particularly in the form of ions, are retained on the concentrate side (dirt side) of the reverse osmosis tank 130.

The reverse osmosis stage 137 may have a recirculation line 135 for recirculating concentrate 105 and a drain 136 for removing or draining concentrate 105. The corresponding explanations for FIG. 1 therefore also apply to FIG. 2. The water with the retained impurities (concentrate 105) on the concentrate side of the reverse osmosis tank 130 is thus, depending on the operating mode, either discarded via the drain 136 with the then open solenoid valve 106 or, when the solenoid valve 106 is closed, fed via the recirculation line 135 with the needle valve 107 to the suction side of the reverse osmosis pump 103 in order to be fed back into the process there.

In normal operation when the reverse osmosis pump 103 is running, purified water or permeate 108 emerging from the membrane 104 on the permeate side (clean side) is fed via a connecting line 138 into a permeate tank 112, which is part of a second sub-unit, also known as the permeate stage 139 or permeate unit. This process takes place until a desired maximum fill level of permeate 108, which is monitored by sensors, is reached in the permeate tank 112. For example, a pressure sensor 113 hydraulically connected to the bottom level of the permeate tank 112 is used to signal that the permeate tank 112 is sufficiently full. The fill level in the permeate tank 112 is calculated using the measured hydrostatic pressure of the permeate column and the known tank geometry by a system controller 150 or control unit (shown here purely schematically). Once the maximum fill level in the permeate tank 112 has been reached, the reverse osmosis pump 103 in the reverse osmosis stage 137 is switched off by the system control unit 150 until the permeate level in the permeate tank 112 falls below a preset, sensor-monitored minimum fill level. This monitoring can also be carried out by means of the pressure sensor 113. In other words, the reverse osmosis pump 103 is controlled (i.e. switched on and off) in such a way that the permeate level in the permeate tank 112 remains within a predetermined range.

The measurement of the fill level in the permeate tank 112 can alternatively/additionally be carried out by other conventional sensors and measuring methods.

To prevent the formation of germs, the permeate 108 in the permeate stage 139 is kept in constant motion during operation of the system by circulating it in a circuit by means of a permeate pump 114. For this purpose, a (permeate) circulation line 140 (also referred to as a ring or circular line) is preferably connected to the bottom of the permeate tank 112 and flows back into the permeate tank 112 at the other end, for example at the top of the permeate tank 112. The permeate pump 114, a sterile filter 116 and an overflow valve 110 are connected to the circulation line 140, preferably in this order (viewed in the direction of flow). The sterile filter 116 is preferably a particle filter. It is intended to separate or retain microorganisms that could potentially be in the permeate circuit. As a rule, it has a pore size of 0.2 μm. The permeate pump 114 drives the permeate 108 through the circuit, residual impurities or impurities or germs in the process of forming are retained in the sterile filter 116, and the overflow valve 110 ensures rudimentary pressure control in the circuit by (only) opening above a preset response pressure.

Between the sterile filter 116 and the overflow valve 110, at least one tapping point 134 is connected to the circulation line 140 by means of a coupling 109 for connecting a consumer or consumer.

After the permeate tank 112 is sufficiently filled with permeate 108, the permeate pump 114 starts and circulates the permeate 108 back into the permeate tank 112 via the sterile filter 116 and the overflow valve 110. The overflow valve 110 is used to maintain a desired pressure at the tapping point 134. An associated dialysis machine is connected to coupling 109 and withdraws permeate 108 as needed. As soon as the level of permeate 108 in the permeate tank 112 falls below a defined level, permeate production in the reverse osmosis stage 137 is restarted by switching on the reverse osmosis pump 103 and opening the inlet-side solenoid valve 101.

Between permeate pump 114 and sterile filter 116, an outlet line 141, which is closed by a solenoid valve 115 during normal operation, branches off from the permeate circulation line 140. At the end of permeate supply, the solenoid valve 115 is opened and the remaining permeate 108 is discarded or the permeate side is rinsed. Preferably, not all of the permeate 108 is discharged from the permeate tank 112, but only reduced to a predefined minimum in order to have as little standing water as possible in the system.

The permeate tank 112 conveniently has a tank ventilation with air filter (not shown in the drawing) in order to prevent overpressure or underpressure due to level changes. In addition, during a standby phase, flushing can be carried out with the minimum volume.

Since the flow resistance of the sterile filter 116 is substantially smaller than the flow resistance of the membrane 104 in the system according to FIG. 1, the permeate pump 114 can be dimensioned substantially smaller than the reverse osmosis pump 103 with respect to its pumping capacity, with corresponding energy savings and a lower noise nuisance when the reverse osmosis pump 103 is paused. The constant low-energy recirculation of the permeate 108 in the permeate stage 139 tends to minimize the formation of germs. The system can be implemented in a space-saving manner with comparatively few components, particularly as a stand-alone system.

### Variant Without Sterile Filter or with Alternative Filter Units

In one possible variant, the sterile filter 116 is dispensed with completely. Instead of a sterile filter 116, another filter unit or, for example, a germicidal UV illumination and/or a disinfecting and/or sterilizing heating of the flowing permeate 108 can also be provided (whereby this heating is switched off during dialysis operation). Such measures can be combined as desired.

### Variant with Volume Flow-Controlled Permeate Pump

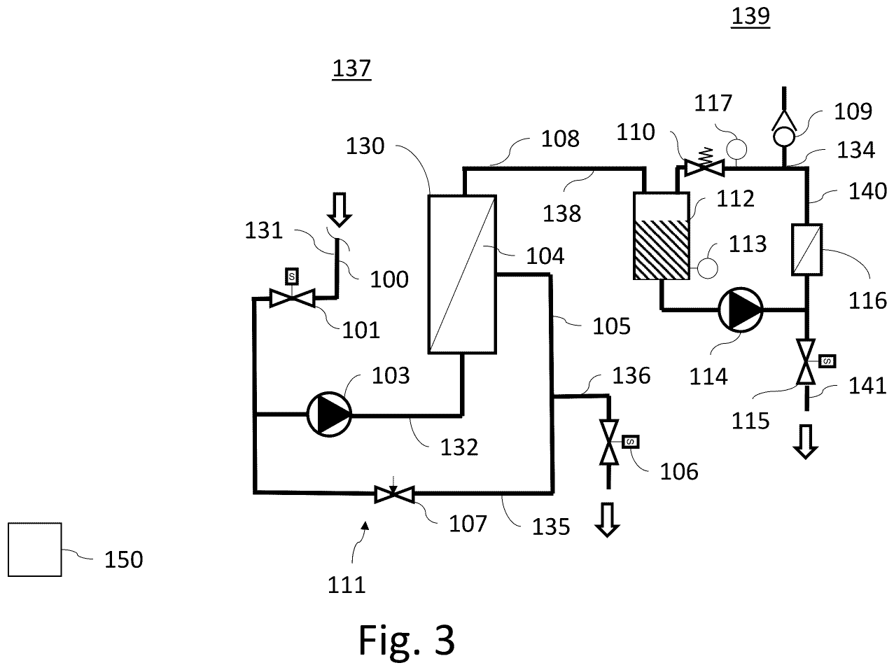

While in the basic version according to FIG. 2 the permeate pump 114 is unregulated (i.e. equipped with pure on/off control), FIG. 3 shows a variant with volume-flow-regulated permeate pump 114. In this case, the control unit (not shown) regulates the speed of the permeate pump 114 to a level such that a predetermined and preferably adjustable flow rate or volume flow of permeate 108 is measured or maintained at the volume flow sensor 117. The volumetric flow sensor 117 is preferably connected between the pick-up point 134 with the coupling 109 and the overflow valve 110 in the permeate circulation line 140 and thereby measures the return flow of permeate 108 per unit of time and volume into the permeate tank 112. This measure can be used to reduce the speed of the permeate pump 114, especially in phases when the connected dialysis machine does not pick up any permeate.

### Variant with Pressure-Controlled Permeate Pump

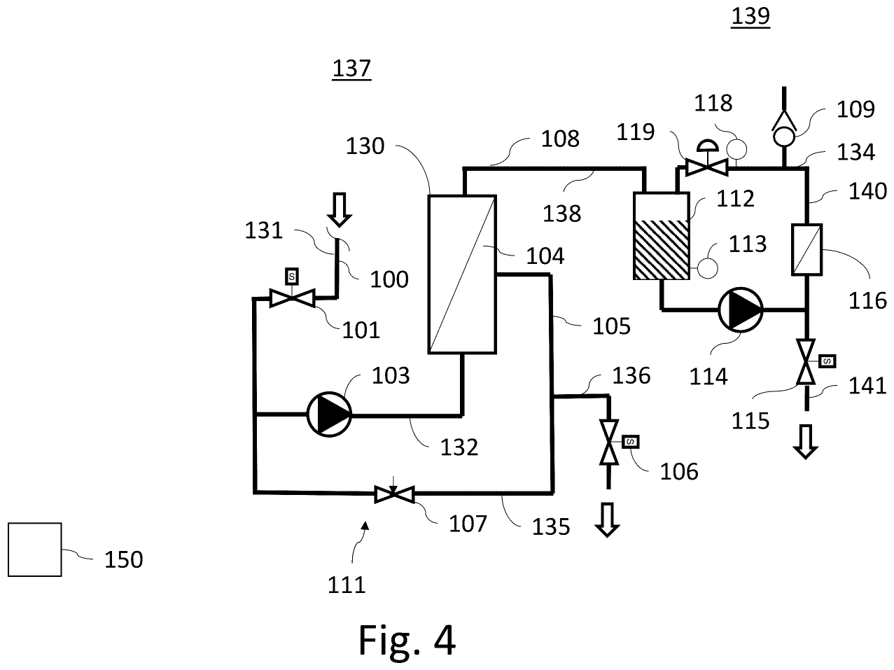

FIG. 4 shows a variant with a pressure-controlled permeate pump 114. Here, the control unit regulates the speed of the permeate pump 114 to a level such that a predetermined and preferably adjustable pressure is measured or maintained at the pressure sensor 118. This allows the speed of the permeate pump 114 to be reduced. The overflow valve 110 from the previously described variants has been replaced by a flow limiter 119 in order to enable the control principle. The pressure sensor 118 is preferably coupled to the permeate circulation line 140 between the tapping point 134 and the flow limiter 119.

### Variant with Storage Tank

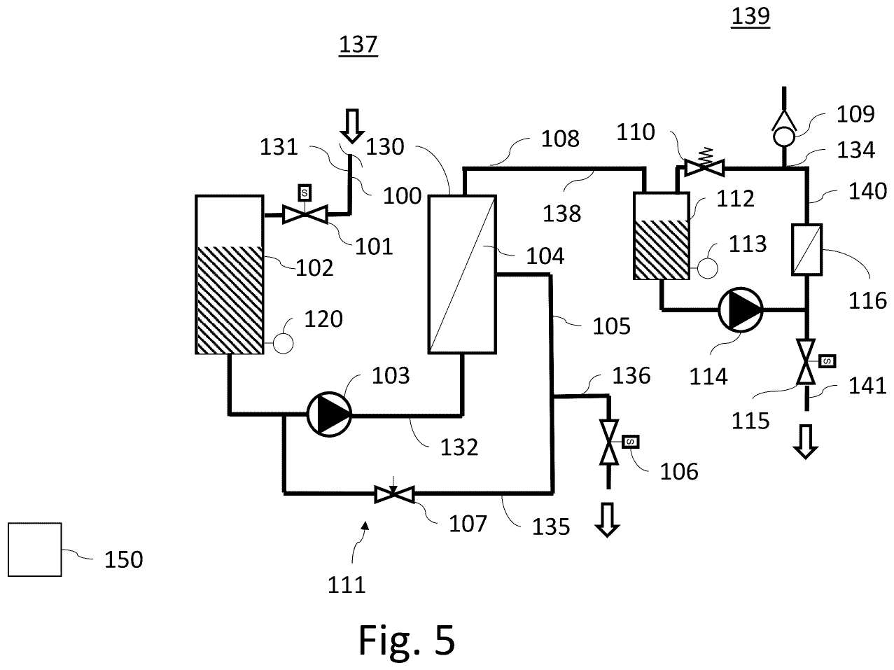

As shown in FIG. 5, the reverse osmosis stage 137 can be extended with a storage tank 102 similar to the prior art. This means that the process input water 100 first flows via the supply line 131 with the solenoid valve 101 into the storage tank 102, which acts as an intermediate storage tank, and from there via the connecting line 132 to the reverse osmosis tank 130. This has the advantage of being able to compensate for a low pressure of the process input water 100, for example in the event of a poor water supply. In this way, dry running of the permeate tank 112 or the dialysis machine connected to the tapping point 134 can be avoided. The volume control (or fill level control) of the process input water 100 in the supply tank 102 preferably takes place via a pressure sensor 120, which measures the hydrostatic pressure of the water column in the supply tank 102—similar to the fill level control in the permeate tank 112 described above.

### Variant with Heater

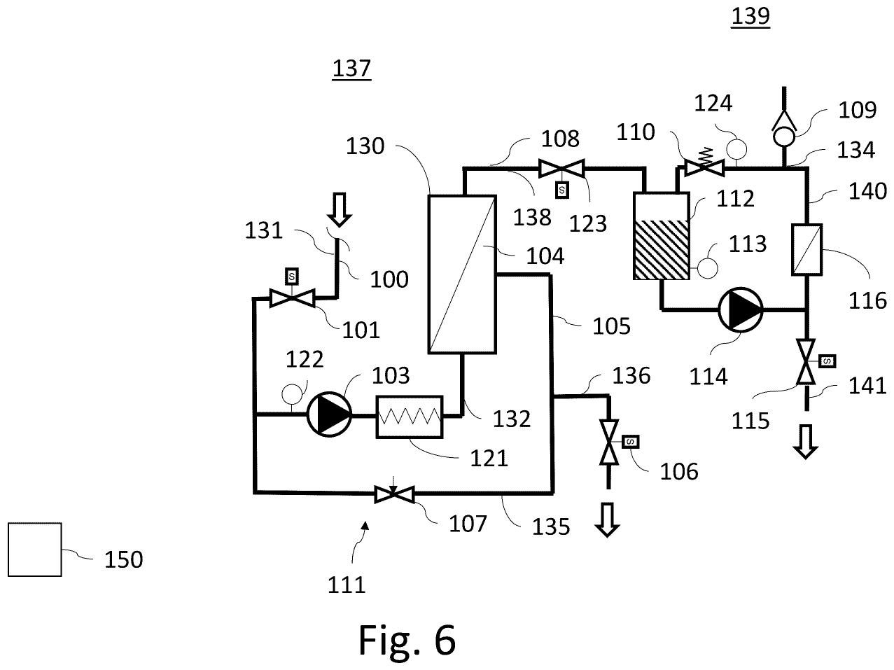

In order to thermally disinfect the entire system, a heater 121, in particular in the form of an electrical heating unit, can be installed in the system, as shown in FIG. 6. Preferably, the heater 121 is connected between the reverse osmosis pump 103 and the reverse osmosis tank 130 in the connecting line 132. In addition, a solenoid valve 123 is installed in the connecting line 138 between the reverse osmosis tank 130 and the permeate tank 112 in order to be able to interrupt the permeate volume flow from the reverse osmosis stage 137 into the permeate stage 139 as required.

During thermal disinfection, the solenoid valve 123 is first closed and the heater 121 is started. The temperature and the heating characteristics can be monitored via the temperature sensor 122, which is preferably coupled to the connecting line 132 upstream of the reverse osmosis pump 103 and measures the temperature of the process input water 100 flowing there. Furthermore, an additional temperature sensor can be provided in the permeate circuit, which measures the disinfection temperature in the permeate circuit.

As soon as the water has been heated accordingly and leaves the reverse osmosis tank 130 in the form of permeate 108, the solenoid valve 123 is opened to convey the water to the permeate stage 139. Alternatively, the solenoid valve 123 can be opened at intervals to keep the rate of temperature change lower by mixing in colder process input water 100.

In the permeate stage 139, the heated water is circulated accordingly and monitored via the temperature sensor 124, which is preferably coupled to the permeate circulation line 140 between the tapping point 134 and the overflow valve 110 (alternatively the flow limiter 119).

Cooling after hot disinfection takes place by discarding the hot water through the outlets controlled by solenoid valves 106 and 115. The discarded water is replaced by colder process inlet water 100.

Disinfection of the stages (reverse osmosis stage 137 and permeate stage 139) is preferably carried out in parallel, but can also be carried out one after the other. In the latter case, however, the disinfection process initially only takes place partially in the reverse osmosis stage, as there is initially no flow on the permeate side.

### Variant with Permeate Recirculation

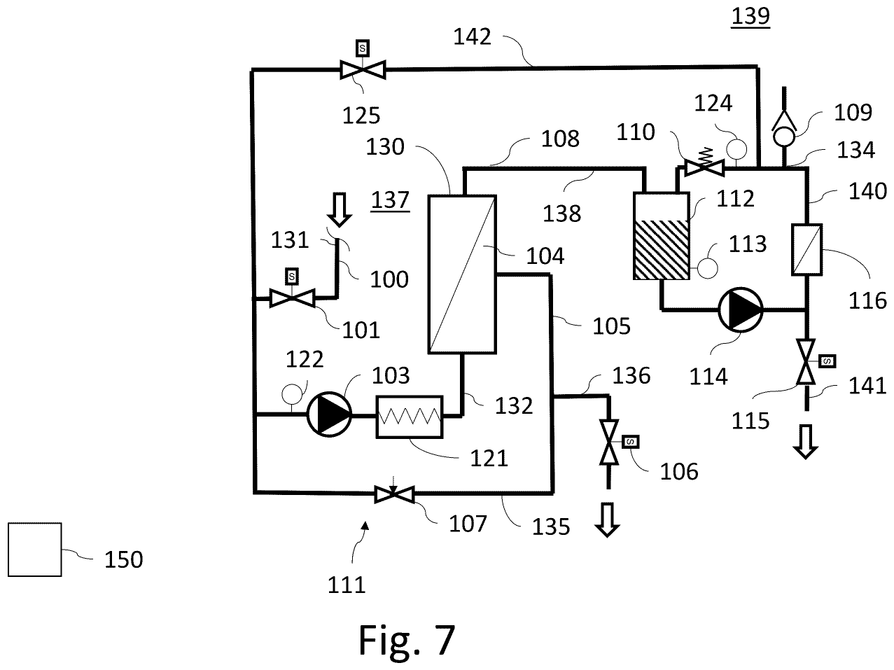

FIG. 7 shows a device based on the variant according to FIG. 6, in which the permeate 108 heated for disinfection by means of heater 121 can be fed from the permeate circuit in the permeate stage 139 via a return line 142 (with open solenoid valve 125) back into the reverse osmosis stage 137. For this purpose, the return line 142 provided with the solenoid valve 125 preferably branches off from the permeate circulation line 140 between the tapping point 134 and the overflow valve 110 (alternatively the flow limiter 119) and opens into the connecting line 132 parallel to the feed line 131, upstream of the reverse osmosis pump 103. This has the advantage that thermal disinfection can be carried out more evenly in the system. Likewise, the reverse osmosis stage 137 can be flushed in standby mode without drawing new process input water 100 via the supply line 131 with the solenoid valve 101.

The return of the permeate 108 from the permeate stage 139 to the reverse osmosis stage 138 is only provided for hot disinfection operation. As in all other variants described, in normal operation, in which normal-temperature permeate 108 is provided at the tapping point 134, the permeate 108 is advantageously not returned from the permeate stage 139 to the reverse osmosis stage 137.

### Variant with Pressure Expansion Vessel

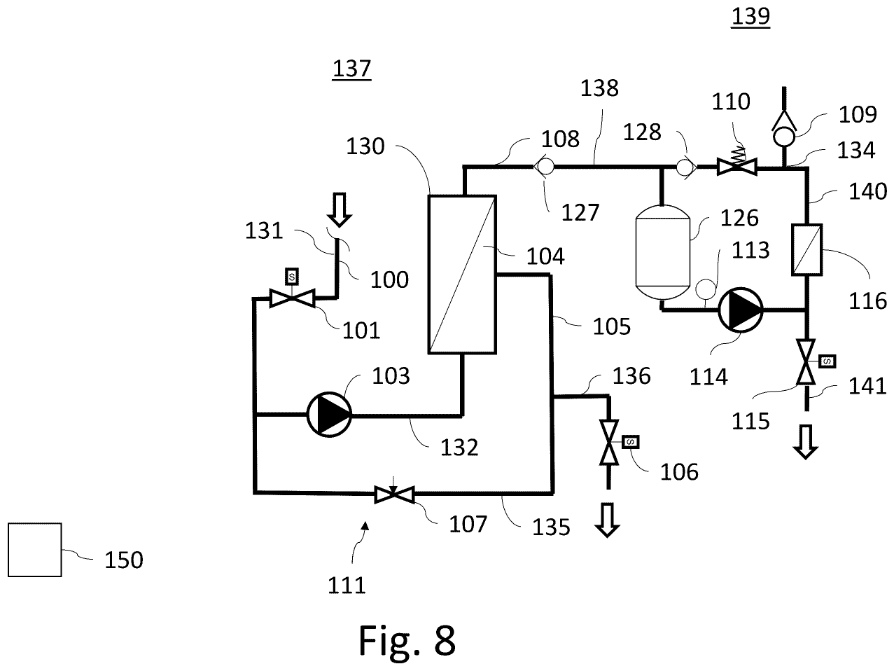

In the variant of FIG. 8, which is based on the basic version according to FIG. 2, a device can be seen which has a pressure expansion vessel 126, also known as a pressure equalization vessel, instead of a conventional permeate tank 112. This is filled with permeate 108 from the reverse osmosis stage 137 until the pressure sensor 113 coupled to the outlet of the pressure expansion vessel 126 exceeds a preset threshold value. The non-return valve 127 in the connecting line 138 between reverse osmosis stage 137 and permeate stage 139 prevents the pressure expansion vessel 126 from forcing permeate 108 back into the membrane 104. The non-return valve 128 in the permeate circulation line 140, preferably in the line section between the overflow valve 110 (alternatively the flow limiter 119) and the pressure expansion vessel 126, prevents an inverse volume flow therein during an operating phase with the reverse osmosis pump 103 switched on or by the action of the pressure expansion vessel 126.

### Variant with Venturi Nozzle

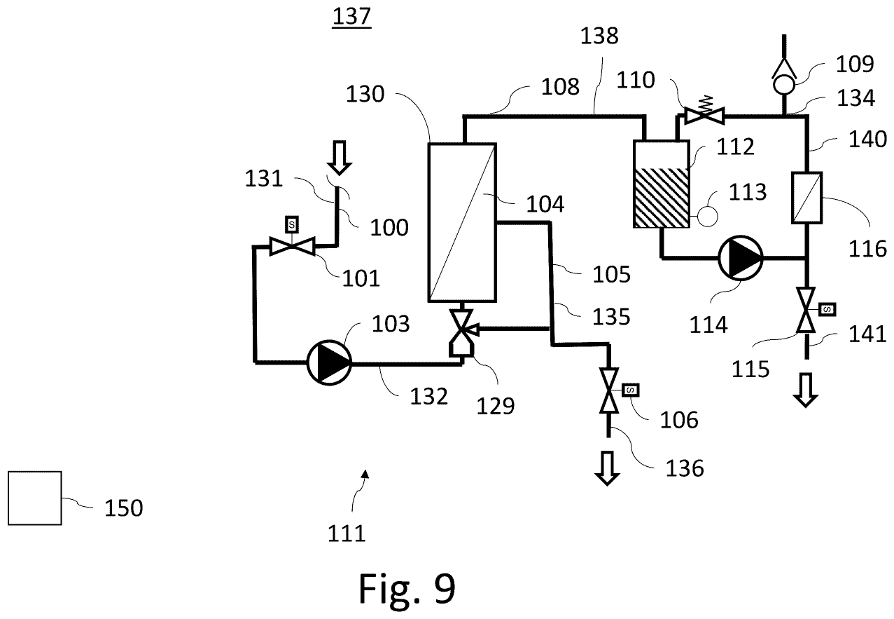

In the variant of FIG. 9, which is based on the basic version according to FIG. 2, a device can be seen which uses a Venturi nozzle 129 connected downstream of the reverse osmosis pump 103 in the connecting line 132 in order to recirculate the concentrate 105 in the reverse osmosis stage 137. The Venturi nozzle 129 replaces the needle valve 107 from the preferred embodiment of FIG. 2. The mode of operation is such that the driving jet of process input water 100 in the connecting line 132 draws in and entrains the concentrate fed at the throat of the Venturi nozzle 129 from the recirculation line 135. The venturi nozzle 129 can have an adjustable nozzle block in order to regulate the volume flow. The Venturi variant represents a form of energy recovery, as the pressure is not lost via the needle valve as is otherwise the case, but is fed in again directly via the Venturi effect on the pressure side.

### Variant with Microbacterially Effective Sterile Filter

The sterile filter 116 can be designed to retain microbacteria in order to retain not only particles but also bacteria or endotoxins.

All the variants mentioned can be combined with each other in any way, provided they are not physically mutually exclusive or build on each other. In particular, all variants can be combined with the basic version according to FIG. 2.

### LIST OF REFERENCE SYMBOLS

100—Process input water
101—Solenoid valve
102—Feed tank
103—Reverse osmosis pump
104—Membrane
105—Concentrate
106—Solenoid valve
107—Needle valve
108—Permeate
109—Coupling
110—Overflow valve
111—Reverse osmosis system
112—Permeate tank
113—Pressure sensor
114—Permeate pump
115—Solenoid valve
116—Sterile filter
117—Volume flow sensor
118—Pressure sensor
119—Flow limiter
120—Pressure sensor
121—Heater
122—Temperature sensor
123—Solenoid valve
124—Temperature sensor
125—Solenoid valve
126—Pressure expansion vessel
127—Non-return valve
128—Non-return valve
129—Venturi nozzle
130—Reverse osmosis tank
131—Supply line
132—Connecting line
133—Ring line
134—Tapping point
135—(Concentrate) recirculation line
136—Drain
137—Reverse osmosis stage
138—Connecting line
138—Permeate stage
140—(Permeate) circulation line
141—Outlet line
142—Return line
143—Inlet
144—Outlet
150—System controller

1. A water treatment plant for production of permeate, the water treatment plant comprising:
a reverse osmosis stage with a piping system having a supply line for process input water, a reverse osmosis pump, and a reverse osmosis tank, the reverse osmosis tank having an inlet for process input water coming from the reverse osmosis pump, a membrane and an outlet for permeate; and
a permeate stage with a line system forming a circuit, the permeate stage having a permeate tank, a permeate pump and a tapping point for connecting at least one consumer or user of permeate,
the reverse osmosis stage and the permeate stage being connected to one another via a connecting line in such a way that the reverse osmosis stage feeds the permeate stage with permeate, and the circuit of the permeate stage comprises a sterile filter.

2. The water treatment plant according to claim 1, wherein the sterile filter is arranged downstream of the permeate pump and upstream of the permeate tank.

3. The water treatment plant according to claim 2, wherein the tapping point is arranged downstream of the sterile filter and upstream of the permeate tank.

4. The water treatment plant according to claim 1, wherein an overflow valve or a flow limiter is arranged between the tapping point and the permeate tank.

5. The water treatment plant according to claim 1, further comprising a system controller that switches the reverse osmosis pump on and off as a function of a fill level in the permeate tank.

6. The water treatment plant according to claim 5, wherein the system controller is configured to calculate the fill level based on hydrostatic pressure.

7. The water treatment plant according to claim 1, wherein the permeate pump is pressure-controlled as a function of pressure in the circuit.

8. The water treatment plant according to claim 7, wherein pressure in the circuit is measured by a pressure sensor arranged in the circuit between the tapping point and a flow limiter arranged upstream of the permeate tank.

9. The water treatment plant according to claim 1, wherein the permeate pump is volume flow controlled as a function of a flow rate through the circuit.

10. The water treatment plant according to claim 9, further comprising a volumetric flow sensor arranged in the circuit between the tapping point and an overflow valve arranged upstream of the permeate tank.

11. The water treatment plant according to claim 1, wherein the permeate tank is a pressure expansion vessel.

12. The water treatment plant according to claim 1, wherein the connecting line is designed such that all permeate production of the reverse osmosis stage is fed into the circuit of the permeate stage.

13. The water treatment plant according to claim 1, wherein the reverse osmosis stage comprises a heater for the process input water, the heater operating as a flow heater.

14. The water treatment plant according to claim 13, wherein the heater is arranged between the reverse osmosis pump and the reverse osmosis tank.

15. The water treatment plant according to claim 1, wherein the reverse osmosis stage comprises a recirculation line for concentrate retained by the membrane.

16. The water treatment plant according to claim 1, wherein the reverse osmosis stage comprises a storage tank for temporarily storing process input water.

17. A method for producing permeate, the method comprising the steps of:
pressing process input water through a membrane using a reverse osmosis pump to create permeate according to principles of reverse osmosis;
feeding the permeate into a permeate tank;
circulating the permeate with a permeate pump in a circuit connected to the permeate tank; and
passing the permeate in the circuit through a sterile filter.

18. The method according to claim 17, further comprising the step of connecting consumers or users of permeate to the circuit.

19. The method according to claim 17, further comprising the step of switching off the reverse osmosis pump when a level of permeate in the permeate tank exceeds a defined value.

20. The method according to claim 17, further comprising the step of controlling the permeate pump as a function of pressure in the circuit.

21. The method according to claim 20, wherein the pressure is measured by a pressure sensor arranged in the circuit between a tapping point for consumers or users of permeate and a flow limiter arranged upstream of the permeate tank.

22. The method according to claim 17, further comprising the step of controlling the permeate pump as a function of a flow rate through the circuit.

23. The method according to claim 22, wherein the flow rate is measured by a volumetric flow sensor arranged in the circuit between a tapping point for consumers or users of permeate and an overflow valve arranged upstream of the permeate tank.
